(19) United States
(12) Patent Application Publication
(10) Pub. No.: US 2006/0177944 A1
(43) Pub. Date: Aug. 10, 2006

(54) RI-labeled compound synthesizing system

(75) Inventor: Motoji Sasaki, Kamakura-shi (JP)

Correspondence Address: SQUIRE, SANDERS & DEMPSEY L.L.P., 14TH FLOOR, 8000 TOWERS CRESCENT, TYSONS CORNER, VA, 22182, US

(73) Assignee: Sumitomo Heavy Industries, Ltd.

(21) Appl. No.: 11/133,447

(22) Filed: May 20, 2005

(30) Foreign Application Priority Data
May 25, 2004 (JP) P2004-155136

(51) Int. Cl. G01N 33/543; B32B 27/04

(52) U.S. Cl. 436/518; 422/159

(57) ABSTRACT

An RI-labeled compound synthesizing system comprises an RI material synthesizing section that synthesizes a labeled precursor by using a radioisotope; a plurality of RI compound manufacturing sections into which the labeled precursor and a reagent are introduced and in which a radioisotope-labeled compound is manufactured; and a switching device that selects an RI compound manufacturing section from among the plurality of RI compound manufacturing sections into which the labeled precursor is introduced.

### BACKGROUND OF THE INVENTION

1. Field of the Invention

The present invention relates to an RI (radioisotope)-labeled compound synthesizing system.

2. Related Background Art

Compounds labeled with radioisotopes (RI compounds) are used for PET (positron emission tomography) in hospitals and the like, for example. RI-compound synthesizing apparatus for manufacturing an RI compound synthesize a labeled precursor by causing a radioisotope (RI) to react with a predetermined material in an RI material synthesizing section, and manufacture the RI compound by using the labeled precursor in an RI compound manufacturing section. In such an RI compound synthesizing apparatus, one RI compound manufacturing section is provided for one RI material synthesizing section (see, for example, Japanese Patent Application Laid-Open No. 2003-21696).

### SUMMARY OF THE INVENTION

The above-mentioned apparatus have been required to manufacture RI compounds continuously with less downtime in order to fulfill demands such as PET, for example.

For solving such a problem, it is an object of the present invention to provide an RI-labeled compound synthesizing system which can continuously manufacture an RI compound.

In one aspect, the present invention provides an RI-labeled compound synthesizing system comprising an RI material synthesizing section that synthesizes a labeled precursor by using a radioisotope; a plurality of RI compound manufacturing sections into which the labeled precursor and a reagent are introduced and in which a radioisotope-labeled compound is manufactured; and a switching device that selects an RI compound manufacturing section from among the plurality of RI compound manufacturing sections into which the labeled precursor is introduced.

Since a plurality of RI compound manufacturing sections are provided for one RI material synthesizing section, this RI-labeled compound synthesizing system can successively utilize a plurality of RI compound manufacturing sections by switching among the plurality of RI compound manufacturing sections as targets to introduce the labeled precursor. As a consequence, during when one RI compound manufacturing section is replaced or its radioactivity decays, other RI compound manufacturing sections can be utilized.

In another aspect, the present invention provides an RI-labeled compound synthesizing system comprising an RI material synthesizing section that synthesizes a labeled precursor by using a radioisotope; a plurality of compound manufacturing sections into which the labeled precursor and a reagent are introduced and in which a radioisotope-labeled compound is manufactured; a common flow path extending from the RI material synthesizing section; a plurality of branched flow paths respectively extending from the plurality of RI compound manufacturing sections; and a switching device disposed between the plurality of branched flow paths and the common flow path, the switching device connecting a selected branched flow path from among the plurality of branched flow paths to the common flow path.

In still another aspect, the present invention provides an RI-labeled compound synthesizing method comprising synthesizing a labeled precursor by using a radioisotope in an RI material synthesizing section; manufacturing a radioisotope-labeled compound by introducing the synthesized labeled precursor and a reagent into a first RI compound manufacturing section; and replacing the first RI compound manufacturing section with new one during when manufacturing a radioisotope-labeled compound by introducing the synthesized labeled precursor and a reagent into a second RI compound manufacturing section after completing the manufacture of the radioisotope-labeled compound in the first RI compound manufacturing section.

The present invention will become more fully be understood from the detailed description given hereinbelow and the accompanying drawings. They are given by way of illustration only, and thus are not to be considered as limiting the present invention.

### BRIEF DESCRIPTION OF THE DRAWINGS

FIG. 1 is a schematic front view showing a methionine synthesizing system in accordance with an embodiment of the present invention;

FIG. 2 is a schematic right side view showing the methionine synthesizing system cut along the line II-II of FIG. 1;

FIG. 3 is a schematic view showing a methyl iodide synthesizing apparatus shown in FIG. 1;

FIG. 4 is a schematic view showing the methyl iodide synthesizing apparatus in the case where the six-way valve shown in FIG. 3 is in a first state;

FIG. 5 is a schematic view showing the methyl iodide synthesizing apparatus in the case where the six-way valve shown in FIG. 3 is in a second state; and

FIG. 6 is a block diagram schematically showing connections of RI compound manufacturing apparatus, a path switching device, and a methyl iodide synthesizing apparatus.

### DESCRIPTION OF THE PREFERRED EMBODIMENTS

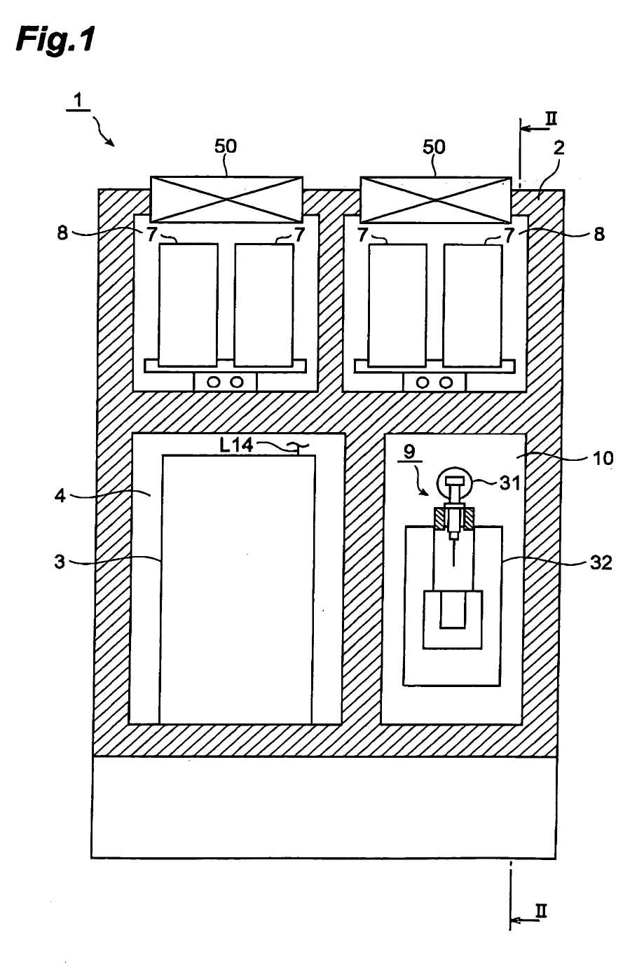

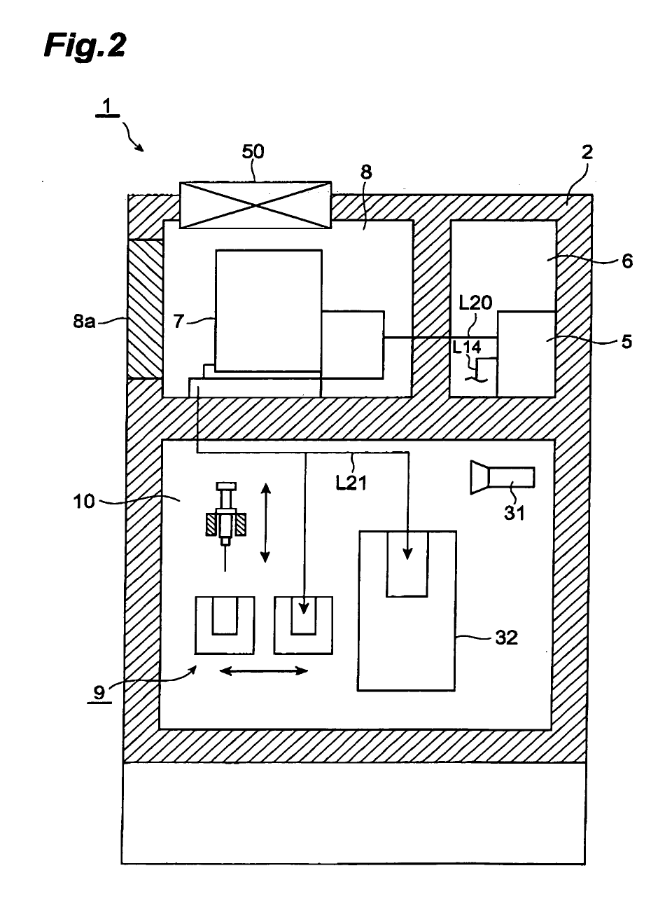

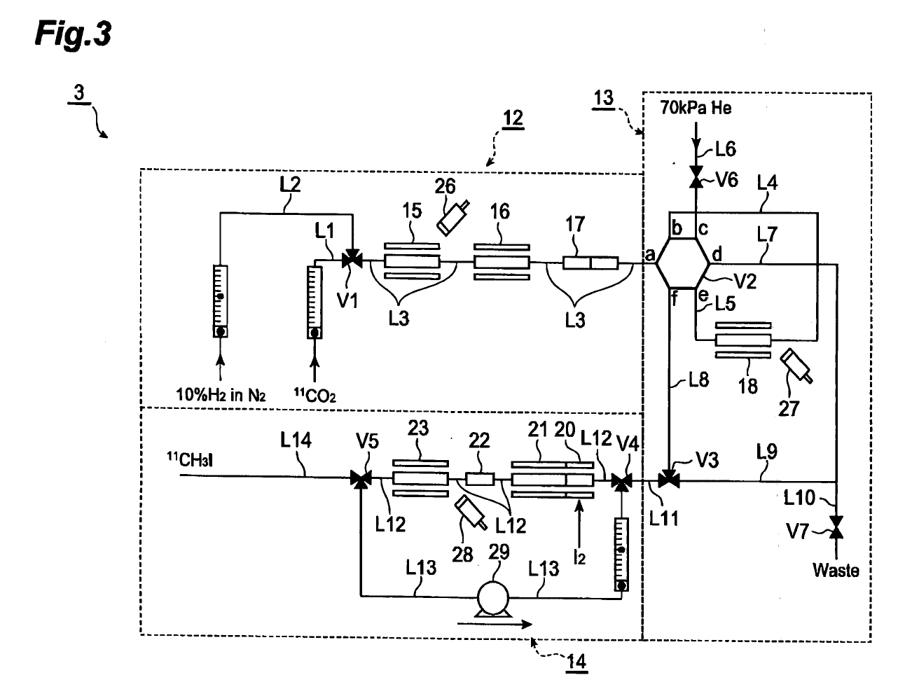

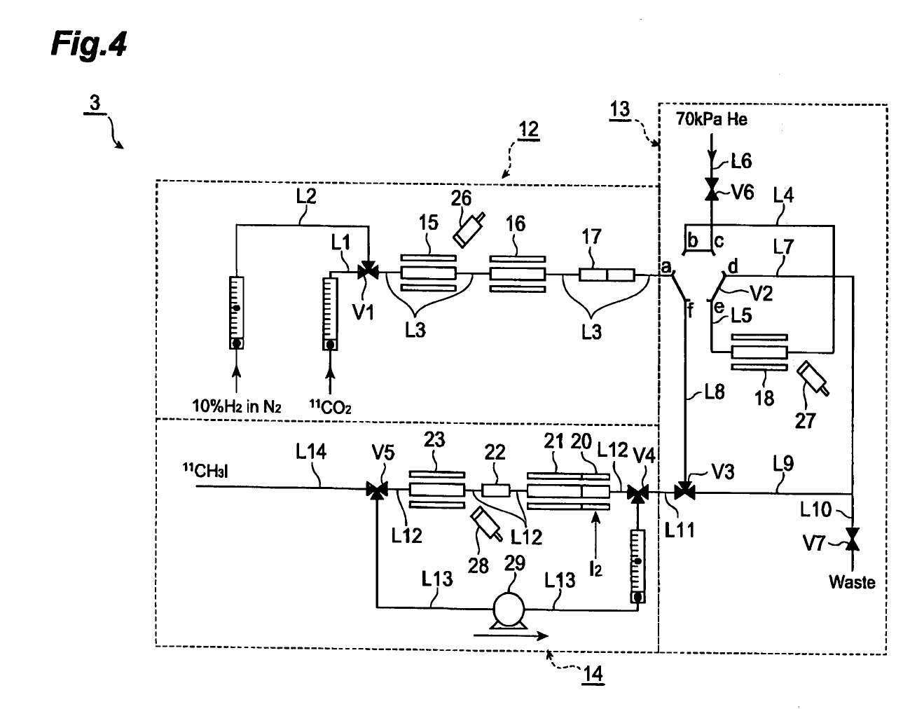

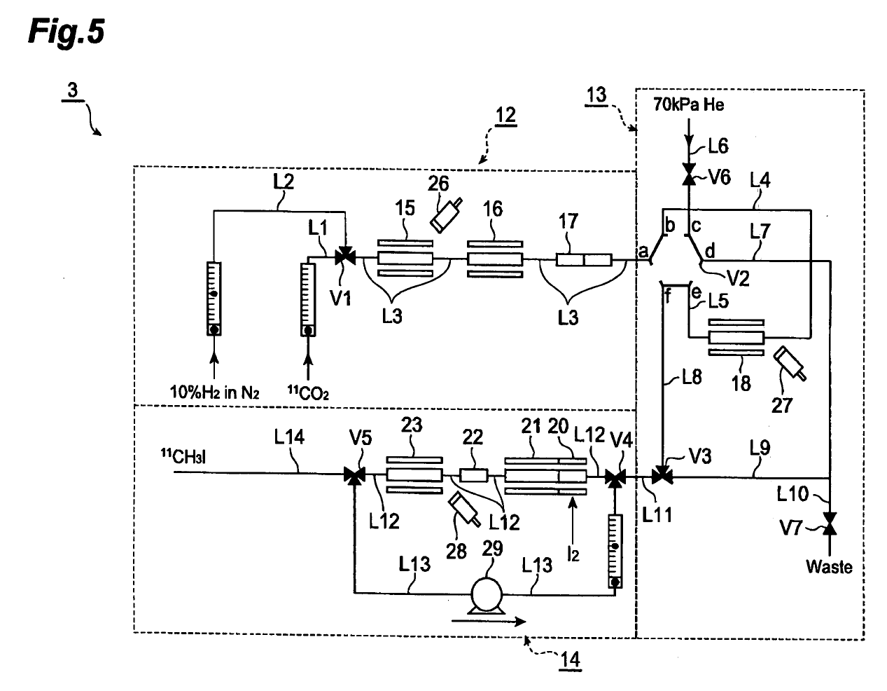

In the following, a preferred embodiment of the RI-labeled compound synthesizing apparatus in accordance with the present invention will be explained with reference to FIGS. 1 to 5. FIGS. 1 and 2 are schematic views showing the methionine synthesizing system in accordance with the embodiment of the present invention. FIG. 3 is a schematic view showing the methyl iodide synthesizing apparatus shown in FIG. 1. FIGS. 4 and 5 are schematic views showing the methyl iodide synthesizing apparatus with respective states of the six-way valve shown in FIG. 3. In the explanation of the drawings, constituents identical or equivalent to each other will be referred to with numerals identical to each other without repeating their overlapping descriptions.

The RI-labeled compound synthesizing system in accordance with this embodiment is a methionine synthesizing system which manufactures [¹¹C]-methionine as a radioactive medicine (including a radioactive pharmaceutical) which is a radioisotope-labeled compound. This radioactive medicine is used for PET in hospitals and the like, for example.

As shown in FIGS. 1 and 2, this methionine synthesizing system 1 is equipped with a radiation shield 2 having a substantially rectangular box form. The radiation shield 2 is formed from a radiation shielding material such as lead, tungsten, or iron, for example, which can block radiation with an appropriate thickness adapted to block radiation, and has such a sealed structure as to prevent radiation from leaking out.

The radiation shield 2 comprises therewithin a plurality of rooms (chambers) each having a sealed structure partitioned with the radiation shielding material. Specifically, the radiation shield 2 comprises a methyl iodide synthesizing chamber (first in-box chamber) 4, a path switching chamber 6, RI compound manufacturing chambers (second in-box chambers) 8, and a quality inspecting chamber (third in-box chamber) 10.

The methyl iodide synthesizing chamber 4 accommodates a methyl iodide synthesizing apparatus (RI material synthesizing section) 3 for synthesizing ¹¹CH₃I which is a labeled precursor. The path switching chamber 6 accommodates a path switching device (switching device) 5 for switching among paths of ¹¹CH₃I synthesized in the methyl iodide synthesizing apparatus 3. The RI compound manufacturing chamber 8 accommodates an RI compound manufacturing apparatus (RI compound manufacturing section) 7 for manufacturing [¹¹C]-methionine by using ¹¹CH₃I synthesized in the methyl iodide synthesizing apparatus 3. The quality inspecting chamber 10 accommodates a radioactive medicine inspecting apparatus (comprising a quality inspecting section and a dispensing section for dispensing a necessary amount) 9 for inspecting the quality of [¹¹C]-methionine manufactured by the RI compound manufacturing apparatus 7.

The methyl iodide synthesizing apparatus 3 will now be explained in detail. As shown in FIG. 3, the methyl iodide synthesizing apparatus 3 roughly comprises a ¹¹CH₄ producing system 12 for reducing ¹¹CO₂ supplied from a cyclotron (not depicted) on the outside of the system with a hydrogen gas, so as to convert it into ¹¹CH₄; a ¹¹CH₄ absorbing system 13 for temporarily absorbing thus obtained ¹¹CH₄; and a ¹¹CH₃I synthesizing system 14 for synthesizing ¹¹CH₃I by causing thus obtained ¹¹CH₄ to react with an iodine gas.

The ¹¹CH₄ producing system 12 comprises a material gas supply line L1 for supplying a material gas containing ¹¹CO₂ into the system; a hydrogen gas supply line L2 for supplying a carrier gas containing a hydrogen gas into the system; a three-way valve V1 for assembling the lines L1, L2 and selecting one of them; an ¹¹CO₂ absorption column 15 for temporarily absorbing ¹¹CO₂ in the material gas; an ¹¹CH₄ conversion column 16 for converting ¹¹CO₂ temporarily absorbed in the ¹¹CO₂ absorption column 15 into ¹¹CH₄; a ¹¹CH₄ refinery column 17 for refining ¹¹CH₄ obtained by the ¹¹CH₄ conversion column 16; and a line L3 connecting the three-way valve V1, ¹¹CO₂ absorption column 15, ¹¹CH₄ conversion column 16, and ¹¹CH₄ refinery column 17 in succession to the ¹¹CH₄ absorption system 13 in a downstream stage.

The ¹¹CO₂ absorption column 15 is filled with an absorbing agent such as Carbosphere (registered trademark), for example, which temporarily absorbs ¹¹CO₂. Disposed on the outside of the ¹¹CO₂ absorption column 15 are a heater/cooler for heating/cooling the ¹¹CO₂ absorption column 15 and a radioactivity monitor 26 for measuring the radioactivity of the ¹¹CO₂ absorption column 15. The absorbing agent for temporarily absorbing ¹¹CO₂ is one which absorbs ¹¹CO₂ at normal temperature and desorbs ¹¹CO₂ when heated.

The ¹¹CH₄ conversion column 16 is filled with a reduction catalyst such as Shimalite Ni (registered trademark), for example, which converts ¹¹CO₂ into ¹¹CH₄ with the aid of the hydrogen gas. Disposed on the outside of the ¹¹CH₄ conversion column 16 is a heater for heating the ¹¹CH₄ conversion column 16.

The ¹¹CH₄ refinery column 17 is filled with an absorbing agent such as Ascarite II (registered trademark) or soda lime, for example, which absorbs unconverted ¹¹CO₂ and the like.

The ¹¹CH₄ absorption system 13 downstream of the ¹¹CH₄ producing system 12 comprises a six-way valve V2, connected to the ¹¹CH₄ refinery column 17, having a plurality of connection ports a to f while being selectable between two types of connection states; a ¹¹CH₄ absorption column 18, connected to the six-way valve V2, for temporarily absorbing ¹¹CH₄; a He supply line L6, equipped with an isolation valve V6, for supplying a He gas into the system; an exhaust line L10, equipped with an isolation valve V7, for discharging an exhaust gas from within the system; and a line L11, connected to a three-way valve V3 which is connected to the exhaust line L10 by way of a line L9, for guiding ¹¹CH₄ from within the system to the ¹¹CH₃I synthesizing system 14 in a downstream stage.

The six-way valve V2 comprises six connection ports a to f. The connection port a is connected to the outlet of the ¹¹C₄ refinery column 17 by way of the line L3. The connection port b is connected to the inlet of the ¹¹CH₄ absorption column 18 by way of a line L4. The connection port c is connected to the He supply line L6. The connection port d is connected to the exhaust line L10 by way of a line L7. The connection port e is connected to the outlet of the ¹¹CH₄ absorption column 18 by way of a line L5. The connection port f is connected to the three-way valve V3 by way of a line L8.

On the other hand, the six-way valve V2 can select any of the first and second states. In the first state, as shown in FIG. 4, the connection ports a, e, and c communicate with the connection ports f, d, and b, respectively. In the second state, as shown in FIG. 5, the connection ports a, c, and e communicate with the ports b, d, and f, respectively.

The ¹¹CH₄ absorption column 18 is filled with an absorbing agent such as Carbosphere (registered trademark), for example, which temporarily absorbs ¹¹CH₄. Disposed on the outside of the ¹¹CH₄ absorption column 18 are a heater/cooler for heating/cooling the ¹¹CH₄ absorption column 18 and a radioactivity monitor 27 for measuring the radioactivity of the ¹¹CH₄ absorption column 18.

The ¹¹CH₃I synthesizing system 14 downstream of the ¹¹CH₄ absorption system 13 comprises a three-way valve V4 connected to the line L11; an iodine column 20 for mixing ¹¹CH₄ with an iodine gas; an ¹¹CH₃I synthesis column 21 for causing the iodine gas evaporated by the iodine column 20 and ¹¹CH₄ to react with each other so as to synthesize ¹¹CH₃I; a ¹¹CH₃I refinery column 22 for refining ¹¹CH₃I; a ¹¹CH₃I absorption column 23 for temporarily absorbing ¹¹CH₃I; a line L12 connecting the three-way valve V4, iodine column 20, ¹¹CH₃I synthesis column 21, and ¹¹CH₃I refinery column 22 in succession; a three-way valve V5 connected to the line L12; a circulating line L13 connecting the three-way valves V5, V4 to each other; a circulating pump 29 placed in the circulating line L13; and a ¹¹CH₃I line L14, connected to the three-way valve V5, for transferring thus synthesized ¹¹CH₃I.

The iodine column 20 is filled with solid iodine. Disposed on the outside of the iodine column 20 is a heater for heating the iodine column 20.

The ¹¹CH₃I synthesis column 21 is constructed by a glass material, for example. Disposed on the ¹¹CH₃I synthesis column 21 is a heater for heating the ¹¹CH₃I synthesis column 21.

The ¹¹CH₃I refinery column 22 is filled with an absorbing agent such as Ascarite II (registered trademark), for example, which absorbs unreacted ¹¹CO₂ and impurities.

The ¹¹CH₃I absorption column 23 is filled with an absorbing agent such as Porapak N, for example, which temporarily absorbs ¹¹CH₃I. Disposed on the outside of the ¹¹CH₃I absorption column 23 are a heater/cooler for heating/cooling the ¹¹CH₃I absorption column 23 and a radioactivity monitor 28 for measuring the radioactivity from the ¹¹CH₃I absorption column 23. The absorbing agent for temporarily absorbing ¹¹CH₃I is one which absorbs ¹¹CH₃I at normal temperature and desorbs ¹¹CH₃I when heated.

In particular, the radiation shield 2 in this embodiment comprises two RI compound manufacturing chambers 8 as shown in FIG. 1. Each RI compound manufacturing chamber 8 accommodates therewithin two RI compound manufacturing apparatus 7 connected to the methyl iodide synthesizing apparatus 3. As shown in FIG. 2, the path switching chamber 6 accommodates therewithin the path switching device 5 for selectively switching between the RI compound manufacturing apparatus 7 into which ¹¹CH₃I is introduced.

The path switching device 5 includes a switching valve for selectively switching between a plurality of outlets. The inlet of the switching valve is connected to the ¹¹CH₃I line (common flow path) L14. The plurality of outlets of the switching valve are connected to the plurality of RI compound manufacturing apparatus 7 by way of a plurality of lines (branched flow paths) L20, respectively.

Each RI compound manufacturing apparatus 7 introduces ¹¹CH₃I, so as to manufacture [¹¹C]-methionine. The RI compound manufacturing apparatus 7 comprises a reagent tank filled with a reagent, the reagent, and a reactor or the like for manufacturing a radioactive medicine by using ¹¹CH₃I, for example. The reagent and ¹¹CH₃I may be caused to react with each other in a reaction column such as a path, for example, without using the reactor.

Each RI compound manufacturing chamber 8 accommodating the RI compound manufacturing apparatus 7 is provided with an intake/exhaust system 50 for feeding a clean air into the manufacturing chamber 8 and discharging the air from within the manufacturing chamber 8. The RI compound manufacturing chambers 8 are provided with respective doors 8a through which the RI compound manufacturing apparatus 7 accommodated therewithin can be let in and out.

A radioactive medicine inspecting apparatus 9 accommodated in the quality inspecting chamber 10 is one which dispenses the radioactive medicine manufactured in the RI compound manufacturing apparatus 7 and inspects the quality thereof. The radioactive medicine inspecting apparatus 9 comprises a product collecting container for collecting the radioactive medicine fed by way of a product collecting line L21; a CCD camera 31 for verifying the property and color of the radioactive medicine, whether impurities mingle with the radioactive medicine or not, and the like; a radioactivity measuring apparatus 32 for measuring the radioactivity from the radioactive medicine; a syringe to be filled with the radioactive medicine; and the like.

Operations of thus configured methionine synthesizing system 1 will now be explained with reference to FIGS. 1 to 5. The methyl iodide synthesizing apparatus 3 successively comprises a ¹¹CO₂ absorbing step of concentrating ¹¹CO₂ in the material gas; a ¹¹CH₄ producing step of producing ¹¹CH₄ by using thus obtained ¹¹CO₂; a ¹¹CH₄ absorbing step of temporarily absorbing ¹¹CH₄, so as to isolate and eliminate an unreacted hydrogen gas and the like; and an ¹¹CH₃I synthesizing step of causing thus obtained ¹¹CH₄ and iodine to react with each other, so as to synthesize ¹¹CH₃I.

In the ¹¹CO₂ absorbing step, as shown in FIG. 4, the material gas is introduced into the ¹¹CO₂ absorption column 15 at room temperature by way of the material gas supply line L1 and three-way valve V1, whereby ¹¹CO₂ in the material gas is temporarily absorbed by the ¹¹CO₂ absorption column 15. The material gas rid of ¹¹CO₂ by the absorbing process is discharged to the outside of the system by way of the line L3, six-way valve V2 in the first state, line L8, three-way valve V3, line L9, exhaust line L10, and isolation valve V7.

After the radioactivity monitor 26 verifies that the amount of ¹¹CO₂ absorption in the ¹¹CO₂ absorption column 15 has reached a predetermined value, the material gas supply is stopped.

Subsequently, the three-way valve V1 is switched such that the hydrogen gas supply line L2 communicates with the line L3, and the six-way valve V2 is switched as shown in FIG. 5 so as to place it into the second state, thereby communicating the line L3, six-way valve V2, line L4, ¹¹CH₄ absorption column 8, line L5, six-way valve V2, line L8, three-way valve V3, and lines L9, L10 together.

In the ¹¹CH₄ producing step, a carrier gas which is mainly composed of a nitrogen gas and contains about 10% of a hydrogen gas is introduced into the ¹CO₂ absorption column 15 by way of the hydrogen gas supply line L2, three-way valve V1, and line L3, while the ¹¹CO₂ absorption column 15 is heated with its heater. This heating process desorbs ¹¹CO₂ from the ¹¹CO₂ absorption column 15.

Together with the carrier gas, thus desorbed ¹¹CO₂ is introduced into the ¹¹CH₄ conversion column 16 heated with its heater, so as to come into contact with the reducing catalyst, and thus is converted into ¹¹CH₄ by the hydrogen gas in the carrier gas.

Thus produced ¹¹CH₄ is introduced into the ¹¹CH₄ refinery column 17, whereby unreacted ¹¹CO₂ and the like accompanying ¹¹CH₄ are absorbed by the absorbing agent stored in the ¹¹CH₄ refinery column 17. This absorbing process separates unreacted ¹¹CO₂ and the like from ¹¹CH₄.

In the ¹¹CH₄ absorbing step, ¹¹CH₄ is introduced into the ¹¹CH₄ absorption column 18 in a room-temperature state by way of the line L3, six-way valve V2, and line L4, and is temporarily absorbed by the ¹¹CH₄ absorption column 18. The unreacted hydrogen gas and the like introduced into the ¹¹CH₄ absorption column 18 together with ¹¹CH₄ pass therethrough, so as to be discharged to the outside of the system by way of the line L5, six-way valve V2, line L8, three-way valve V3, line L9, exhaust line L10, and isolation valve V7.

After the radioactivity measurement by the radioactivity monitors 26, 27 verifies that the amount of ¹¹CO₂ absorption in the ¹¹CO₂ absorption column has decreased and that the amount of ¹¹CH₄ in the ¹CH₄ absorption column 18 has reached a predetermined value, the carrier gas supply is stopped.

The ¹¹CH₄ absorbing step includes a purging step of purging the inside of the ¹¹CH₄ absorbing system 13. First, the three-way valve V3 is closed while the isolation valve V6 is opened, and the six-way valve V2 is switched to the first state as shown in FIG. 4.

The He gas supplied from the He supply line L6 in this state passes the isolation valve V6, six-way valve V2, line L4, ¹¹CH₄ absorption column 8, line L5, six-way valve V2, line L7, exhaust line L10, and isolation valve V7, thereby discharging the hydrogen gas and the like remaining in these lines, valves, and ¹¹CH₄ absorption column 18 to the outside of the system. After a predetermined amount of He gas is supplied, the three-way valve V3 is switched such that the lines L9 and L11 communicate with each other, and the isolation valve V7 is closed. Thus, the lines L7, L9, and L11 are communicated together.

Subsequently, the ¹¹CH₄ absorption column 18 is heated with its heater. This heating process desorbs ¹¹CH₄ from the ¹¹CH₄ absorption column 18.

In the ¹¹CH₃I synthesizing step, thus desorbed ¹¹CH₄ is transferred by the He gas, so as to be introduced into the ¹¹CH₃I synthesizing system 14 by way of the line L5, six-way valve V2, lines L7, L9, three-way valve V3, line L11, and three-way valve V4. Thus introduced ¹¹CH₄ passes the line L12, iodine column 20, ¹¹CH₃I synthesis column 21, ¹¹CH₃I refinery column 22, ¹¹CH₃I absorption column 23, three-way valve V5, circulating line L13, and circulating pump 29, so as to return to the three-way valve V4, thereby circulating these lines, valves, and columns.

In the state where ¹¹CH₄ thus circulates through the ¹¹CH₃I synthesizing system 14, the iodine column 20 is heated with its heater. This heating process evaporates iodine, whereby thus obtained iodine gas mingles with ¹¹CH₄.

The mixture of ¹¹CH₄ and iodine gas is introduced into the ¹¹CH₃I synthesis column 21 and is heated with its heater. This heating process causes ¹¹CH₄ and the iodine gas to react with each other, so as to synthesize ¹¹CH₃I.

Thus synthesized ¹¹CH₃I is introduced into the ¹¹CH₃I refinery column 22, whereby unreacted ¹¹CO₂ and the like accompanying ¹¹CH₃I are absorbed by the absorbing agent stored in the ¹¹CH₃I refinery column 22. This absorbing process separates ¹¹CO₂ and the like from ¹¹CH₃I.

¹¹CH₃I rid of ¹¹CO₂ is introduced into the ¹¹CH₃I absorption column 23 at room temperature, whereby ¹¹CH₃I is temporarily absorbed by the ¹¹CH₃I absorption column 23. Unreacted ¹¹CH₄ introduced into the ¹¹CH₃I absorption column 23 together with ¹¹CH₃I passes therethrough and keeps circulating, so as to be introduced into the iodine column 20 again, whereby the mixing with the iodine gas, synthesis reaction, and the like are repeated as mentioned above.

After the radioactivity monitor 28 verifies that the amount of ¹¹CH₃I absorption in the ¹¹CH₃I absorption column 23 has reached a predetermined value, the circulating pump 29 is stopped so as to terminate the circulation, and the three-way valves V4, V5 are switched, so as to communicate the lines L11, L12, and L14 together.

Subsequently, the ¹¹CH₃I absorption column 23 is heated with its heater. This heating process desorbs ¹¹CH₃I from the ¹¹CH₃I absorption column 23. Thus desorbed ¹¹CH₃I is transferred by the He gas introduced from the He supply line L6, so as to pass the lines L12, L14, thereby being collected as a product on the outside of the system. This yields ¹¹CH₃I. Thus obtained ¹¹CH₃I passes the ¹¹CH₃I line L14, path switching device 5, and line L20, so as to be supplied to the RI compound manufacturing apparatus 7.

In the RI compound manufacturing apparatus 7, thus introduced ¹¹CH₃I and the predetermined reagent stored in the reagent tank are regulated by an electromagnetic valve in terms of their flows so as to be introduced into the reactor by way of predetermined hole paths, and are caused to react with each other, so as to synthesize a reaction product, which is then collected as a radioactive medicine. Consequently, [¹¹C]-methionine as a radioactive medicine is manufactured while using ¹¹CH₃I which is a labeled precursor. The radioactive medicine is supplied to the radioactive medicine inspecting apparatus 9 by way of the line L21.

The above-mentioned inspection is performed in the radioactive medicine inspecting apparatus 9, whereas a small amount of the radioactive medicine is taken out by the syringe, so as to be subjected to other quality inspections by an analyzer or the like on the outside of the system. One passing these quality inspections is considered to be a radioactive medicine which can be administered to human bodies.

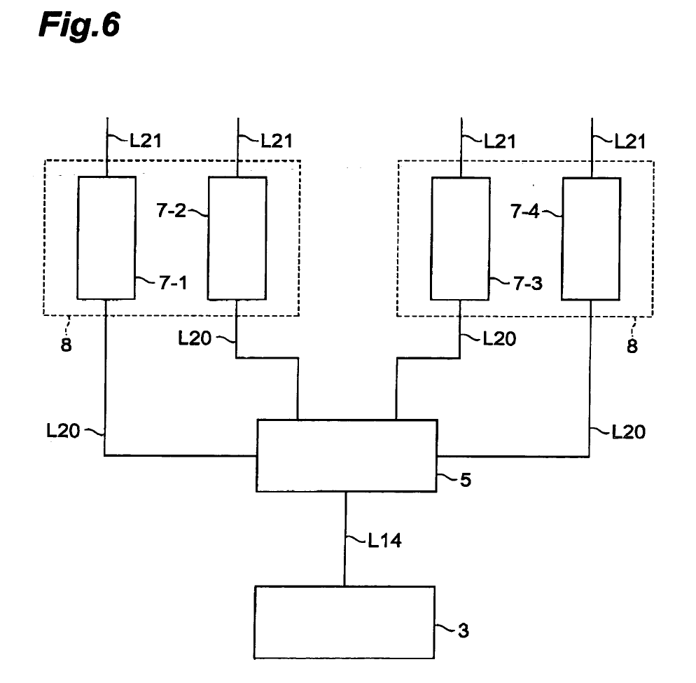

As shown in FIG. 6, when the manufacture of the radioactive medicine in one RI compound manufacturing apparatus 7-1 is completed, the path switching device 5 selectively switches the path, so that ¹¹CH₃I is introduced into the other RI compound manufacturing apparatus 7-2 in the same RI compound manufacturing chamber 8. When the manufacture of the radioactive medicine is completed in the RI compound manufacturing apparatus 7-2, the path switching device 5 switches the path, so that ¹¹CH₃I is introduced into one RI compound manufacturing apparatus 7-3 in another RI compound manufacturing chamber 8. When the manufacture of the radioactive medicine in the RI compound manufacturing apparatus 7-3 is completed, ¹¹CH₃I is introduced into the other RI compound manufacturing apparatus 7-4 in the same RI compound manufacturing chamber 8, and these operations are repeated as necessary.

After the lapse of a predetermined time from the completion of manufacture of the radioactive medicine in the two RI compound manufacturing apparatus 7-1, 7-2 in one RI compound manufacturing chamber 8, at which the radioactivity within this RI compound manufacturing chamber 8 has sufficiently decayed, the door of the RI compound manufacturing chamber 8 is opened, so that the two RI compound manufacturing apparatus 7-1, 7-2 are taken out, whereas other RI compound manufacturing apparatus 7 are accommodated into the RI compound manufacturing chamber 8, so as to manufacture the radioactive medicine again. Thus, the RI compound manufacturing apparatus 7 are exchanged after manufacturing the radioactive medicine. At this time, in parallel with the exchange of the RI compound manufacturing apparatus, the radioactive medicine is manufactured in the RI compound manufacturing apparatus 7-3, 7-4 in another RI compound manufacturing chamber 8.

Thus, in this embodiment, a plurality of RI compound manufacturing apparatus 7 are provided for one methyl iodide synthesizing apparatus 3, whereas the path switching device 5 changes the RI compound manufacturing apparatus 7 to which ¹¹CH₃I is introduced, whereby the other RI compound manufacturing apparatus 7 can be utilized sequentially. As a result, the radioactive medicine can be manufactured continuously. Since a plurality of RI compound manufacturing chambers 8 each accommodating a plurality of RI compound manufacturing apparatus 7 are provided, while replacing the RI compound manufacturing apparatus 7-1, 7-2 in one RI compound manufacturing chamber 8, the RI compound manufacturing apparatus 7-3, 7-4 in another RI compound manufacturing chamber 8 can be utilized. This can provide the methionine synthesizing system 1 which can continuously manufacture the radioactive medicine while keeping a sanitary condition and reducing the radiation dose.

In the methionine synthesizing system 1 of this embodiment, the manufacturing time for the radioactive medicine per cycle in the RI compound manufacturing apparatus 7 is 60 minutes, whereby the total manufacturing time in the compound manufacturing chamber 8 is 120 minutes. Since the half life of the radioisotope ¹¹C contained in this radioactive medicine is 20 minutes, the radioactivity remaining in the apparatus decays to 1/64 to ⅛ in the two cycles of radioactive medicine manufacturing time.

Since this embodiment is equipped with the radiation shield 2 integrally comprising the methyl iodide synthesizing chamber 4, RI compound manufacturing chambers 8, 8, and quality inspecting chamber 10, the methionine synthesizing system 1 can be made smaller.

Though the present invention is specifically explained with reference to its embodiment in the foregoing, the present invention is not limited to the above-mentioned embodiment. For example, though the above-mentioned embodiment relates to the methionine synthesizing system 1 for manufacturing [¹¹C]-methionine, the system may be any of RI-labeled compound synthesizing systems for manufacturing other radioactive medicines such as [¹¹C]-choline, for example, radioactive pharmaceuticals, and RI compounds.

Though the above-mentioned embodiment has a configuration comprising a plurality of RI compound manufacturing chambers 8 each accommodating a plurality of RI compound manufacturing apparatus 7, it may comprise a plurality of RI compound manufacturing chambers 8 each accommodating a single RI compound manufacturing apparatus 7. The RI compound manufacturing chambers 8 each accommodating a single RI compound manufacturing apparatus 7 and the RI compound manufacturing chambers 8 each accommodating a plurality of RI compound manufacturing apparatus 7 may be combined together as well.

From the invention thus described, it will be obvious that the same may be varied in many ways. Such variations are not to be regarded as a departure from the spirit and scope of the invention, and all such modifications as would be obvious to one skilled in the art are intended to be included within the scope of the following claims.

The disclosure of Japanese Patent Application No. 2004-155136 filed on May 25, 2004 including specification, drawings and claims is incorporated herein by reference in its entirety.

What is claimed is:

1. An RI-labeled compound synthesizing system comprising:
an RI material synthesizing section that synthesizes a labeled precursor by using a radioisotope;
a plurality of compound manufacturing sections into which the labeled precursor and a reagent are introduced and in which a radioisotope-labeled compound is manufactured; and
a switching device that selects an RI compound manufacturing section from among the plurality of RI compound manufacturing sections into which the labeled precursor is introduced.

2. An RI-labeled compound synthesizing system according to claim 1, farther comprising a radiation shield that accommodates the RI material synthesizing section and the plurality of RI compound manufacturing sections;
wherein the radiation shield integrally comprises a first box sealed with a radiation shielding material and a second box, sealed with a radiation shielding material, having a door adapted to open and close; and
wherein the RI material synthesizing section is accommodated in the first box, whereas the RI compound manufacturing sections are accommodated in the second box.

3. An RI-labeled compound synthesizing system according to claim 2, wherein the second box includes a plurality of chambers divided by and sealed with the radiation shielding material; and
wherein each of the plurality of chambers has the door.

4. An RI-labeled compound synthesizing system according to claim 1, further comprising a quality inspecting section that inspects a quality of the radioisotope-labeled compound.

5. An RI-labeled compound synthesizing system according to claim 4, further integrally comprising a third box sealed with a radiation shielding material;
wherein the quality inspecting section is accommodated in the third box.

6. An RI-labeled compound synthesizing system according to claim 1, wherein the radioisotope-labeled compound is [¹¹C]-methionine.

7. An RI-labeled compound synthesizing system comprising:
an RI material synthesizing section that synthesizes a labeled precursor by using a radioisotope;
a plurality of compound manufacturing sections into which the labeled precursor and a reagent are introduced and in which a radioisotope-labeled compound is manufactured;
a common flow path extending from the RI material synthesizing section;
a plurality of branched flow paths respectively extending from the plurality of RI compound manufacturing sections; and
a switching device disposed between the plurality of branched flow paths and the common flow path, the switching device connecting a selected branched flow path from among the plurality of branched flow paths to the common flow path.

8. An RI-labeled compound synthesizing system according to claim 7, wherein the switching device includes a switching valve.

9. An RI-labeled compound synthesizing method comprising:
synthesizing a labeled precursor by using a radioisotope in an RI material synthesizing section;
manufacturing a radioisotope-labeled compound by introducing the synthesized labeled precursor and a reagent into a first RI compound manufacturing section; and
replacing the first RI compound manufacturing section with new one during when manufacturing a radioisotope-labeled compound by introducing the synthesized labeled precursor and a reagent into a second RI compound manufacturing section after completing the manufacture of the radioisotope-labeled compound in the first RI compound manufacturing section.
